(19) United States
(12) Patent Application Publication
(10) Pub. No.: US 2017/0128542 A1
(43) Pub. Date: May 11, 2017

(54) NON-AQUEOUS GLUCAGON FORMULATIONS

(71) Applicant: Albireo Pharma, Inc., Boston, MA (US)

(72) Inventors: Ming Li, Yorktown Heights, NY (US); Roderike Pohl, Sherman, CT (US); Robert Feldstein, Yonkers, NY (US); Robert Hauser, Columbia, MD (US); Errol De Souza, Cambridge, MA (US)

(21) Appl. No.: 15/347,169

(22) Filed: Nov. 9, 2016

Related U.S. Application Data
(60) Provisional application No. 62/252,941, filed on Nov. 9, 2015.

(51) Int. Cl. A61K 38/26; A61K 47/34; A61K 9/08; A61K 47/10; A61K 47/14; A61K 47/20

(52) U.S. Cl. CPC A61K 38/26; A61K 47/14; A61K 47/20; A61K 9/08; A61K 47/10; A61K 47/34

(57) ABSTRACT

Stabilized glucagon formulations are provided, in the form of a clear solution. The formulations include glucagon in a non-aqueous diluent and an antioxidant The diluent and antioxidant are selected to provide a glucagon formulation with increased stability when compared to glucagon without the anti-oxidant and/or the diluent and which shows a comparable onset of action and duration of glucose response.

The compositions can be used to treat subjects with very low blood sugar (severe hypoglycemia) that can happen in subjects who have diabetes and use insulin.

### CROSS-REFERENCE TO RELATED APPLICATIONS

This application claims benefit of U.S. Provisional Application No. 62/252,941 filed Nov. 9, 2015, which is incorporated by reference in its entirety.

### FIELD OF THE INVENTION

The invention is generally directed to stabilized glucagon formulations.

### BACKGROUND OF THE INVENTION

Glucagon, a hormone secreted by the pancreas, is a polypeptide consisting of a single chain of 29 amino acids and has a molecular weight of 3,485 Da. Medically, glucagon is used to treat hypoglycemia (characterized by lower than normal blood glucose concentrations). Hypoglycemia is common in Type-1 diabetic patients and insulin users. Mild hypoglycemia causes anxiety, sweating, tremors, palpitations, nausea, and pallor. In severe hypoglycemia, the brain is starved of the glucose it needs for energy, leading to seizures, coma or even death. Severe hypoglycemia is a life-threatening emergency that requires immediate medical intervention, for which the current standard of care is glucagon injection. Glucagon is not absorbed orally and is therefore administered by injection. Upon injection, glucagon stimulates the liver to convert stored glycogen into glucose, which is released into the blood. The onset of action for glucagon occurs 5-20 minutes after injection. The half-life of glucagon in blood is 3 to 6 minutes, which is similar to insulin.

Glucagon has an isoelectric point of 7.1. In aqueous solutions of pH 3 or less, glucagon is initially soluble, but will aggregate to form a gel within an hour. The gelled glucagon consists predominantly of β-sheet fibrils that are induced by the hydrophobicity and the inter- and intra-chain hydrogen bond forming potential of the peptide (Chou, et al. Biochemistry, 14(11):2536-2541 (1975). The aggregated glucagon is not suitable for injection because the gel can clog a hypodermic needle and, if intravenously administered, blood vessels. To slow the aggregation process, an acidic (pH 2-4) formulation is commonly used to maintain glucagon in a relatively aggregation-free state for a short time. Such acidic formulations must be injected immediately after preparation as the glucagon will aggregate (Product Insert for GLUCAGEN® HYPOKIT® for injection [glucagon [rDNA origin]). In addition to its physical instability, glucagon undergoes various types of chemical degradation. In aqueous solution, it rapidly degrades to form several degradation products. At least 16 degradation products of glucagon have been reported with the major degradation pathways being aspartic acid cleavage at positions 9, 15, and 21, and glutaminyl deamidation at positions 3, 20 and 24 (Kirsch, et al., International Journal of Pharmaceutics, 203:115-125 (2000). The chemical degradation of glucagon is rapid and complex. For example, in an acidic solution (pH 2-4) required to dissolve glucagon and prevent its aggregation, about 5-70% of the glucagon decomposes into numerous degradation products within 24 hours at 37° C. (U.S. Patent Application Publication No. 2011/0097386). This instability has limited the medical utility of the currently available glucagon formulations.

Glucagon is indicated for the treatment of severe hypoglycemia. In order to circumvent glucagon's chemical instability, the currently available glucagon drug products (e.g., GLUCAGEN® HYPOKIT® (glucagon hydrochloride) from Novo Nordisk and Glucagon for Injection (rDNA origin) from Eli Lilly and Company) are lyophilized and provided as two-part kits. One part is a vial containing 1 mg (1 unit) of glucagon and 49 mg of lactose in a dry lyophilized solid mass (“cake”) and the other part is a syringe containing a diluent which includes 12 mg/mL glycerin, water and hydrochloric acid. Lyophilization provides an anhydrous environment that keeps glucagon stable by preventing aspartic acid cleavage, glutaminyl deamidation and any water-dependent degradative pathways. To use the glucagon kit, the diluent is first injected from the syringe into the cake-containing vial, which is then gently swirled to dissolve the glucagon. The reconstituted glucagon solution is then drawn back into the same syringe, which is now ready for injection. The pH of this solution is approximately 2.0-3.5. The reconstituted glucagon solution is unstable and the manufacturers recommend it to be used immediately after reconstitution and to discard any unused portion. Thus, each glucagon kit is intended only for a single and immediate use.

The proper use of the two-part glucagon kit requires a complicated multiple-step procedure that includes taking stock of the kit components, removing the cap seal, injecting the diluent into the vial, reconstituting the glucagon cake, withdrawing the glucagon solution, and administering the reconstituted solution. This cumbersome procedure could be difficult even for a normal person to perform. For someone incapacitated by hypoglycemia, the task may be extremely difficult or impossible. A delay in administering timely glucagon rescue therapy could result in death. Sadly, 6-10% of deaths of individuals with Type 1 diabetes are a result of hypoglycemia (Cryer, Diabetes 57(12): 3169-3176 (2008)). Thus, a stable and ready-to-inject liquid glucagon formulation would be highly desirable for emergency hypoglycemia rescue and has the potential to save lives.

Insulin pumps have been widely used by insulin dependent diabetics for over a decade. These pumps provide a continuous flow of insulin to patients. After a meal, the user can manually increase the insulin flow to temporarily cover the post-prandial blood glucose surge, and then dial back to a slow basal maintenance flow. These pumps can be attached directly to the abdominal surface and deliver insulin directly to subcutaneously inserted small needles (e.g., the OMNIPOD® from Insulet Corp.) or can be worn externally in close proximity to the body and deliver insulin via fine tubing through subcutaneously implanted needles (e.g., ONETOUCH PING® (Animas Corp.), PARADIGM®REVEL™ (Medtronic, Inc.), and others). The subcutaneous needles may remain in place for up to a week. A bi-hormonal closed loop pump or a true artificial pancreas is a CGM-linked insulin pump, which is capable of delivering both insulin and glucagon to the patient. A true bi-hormonal pump requires a liquid glucagon formulation that is stable for at least three to seven days at body or near body temperature.

Stabilized glucagon formulations have been developed to prevent glucagon aggregation or gelation, i.e., to address glucagon's physical instability in the solution state. Without reducing chemical degradation to an acceptable level, any glucagon composition will have limited application as a drug product.

To prevent glucagon aggregation, gelation or precipitation, most known formulations employ water-soluble surfactants, detergents or well-known drug solubilizers that dissolve glucagon to form a clear solution. These attempts have included: using up to a six-fold molar excess of cationic or anionic monovalent detergent (Great Britain Patent 1202607); hen egg lysolecithin (Schneider, et al. Biol. Chem., 247: 4986-4991 (1972); lysolecithin (Robinson, et al. Biopolymers, 21: 1217-1228 (1982); micelles of anionic detergent sodium dodecyl sulfate (SDS) at low pH (Wu, et al., Biochemistry, 19:2117-2122 (1978) and SDS micelles at neutral pH (Brown, et al., Biochim. Biophys. Acta, 603: 298-312 (1980); cyclodextrins (Matilainen, et al., J. Pharm Sci., 97(7):2720-9 (2008) and Matilainen, et al., Eur. J. Pharm Sci., 36(4-5):412-20 (2009); lysophospholipids (1-acyl-sn-glycero-3-phosphoate ester of ethanolamine, choline, serine or threonine) or other detergents such as cetyl trimethylammonium bromide (CTAB) and SDS, etc. (European Patent 1061947); and lysophospholipid-sugar combinations (US Patent Application 2011/0097386).

There is still a need for glucagon formulations with improved physical and chemical stability.

It is therefore an object of the present invention to provide a glucagon that is stable as a clear solution for at least seven days at 37° C.

### SUMMARY OF THE INVENTION

Stabilized glucagon formulations are provided, in the form of a clear solution. The formulations include glucagon in a non-aqueous diluent and an antioxidant. The diluent and antioxidant are selected to provide a glucagon formulation with increased stability when compared to glucagon without the anti-oxidant and/or the diluent and which shows a comparable onset of action and duration of glucose response, as to GLUCAGEN®. The combination stabilizes the glucagon used at a concentration between 0.5-4 mg/mL, preferably, between 1-2 mg/mL, and at a pH range of 2-4.5 or 8-9. Preferably, the pH is acidic, between 2-4. Additional excipients may be added to stabilize the formulation or control gelation or viscosity. In some embodiments, the formulations additionally include a surfactant. The formulation may also be in the form of a microemulsion or liposomes.

In the preferred embodiment shown in the examples, the stabilized glucagon solution contains a non aqueous diluent such as propylene glycol (PG) and diethylene glycol monoethyl ether (TRANSCUTOL® (“TC”)) and an antioxidant. The PG and TC are present the diluent in a ratio ranging from 1:4 to 4:1, preferably, in a ratio ranging from 1:1 to 1:2. A most preferred ratio for PG and TC is 1:1. Preferred antioxidants include propyl gallate (C8 and C12; 0.01-10 mg/mL), butylated hydroxylanisole (0.001-10 mg/ml), butylated hydroxytoluene (0.001-10 mg/mL), d-α-tocopheryl polyethylene glycol 1000 succinate (TPGS) (2-20 mg/mL) and methionine (0.05-10 mg/ml), alone, or in combination. The formulations can include water in a concentration ranging from 0-5%, preferably, from 1-3%.

The compositions can be used to treat subjects with very low blood sugar (severe hypoglycemia) that can happen in subjects who have diabetes and use insulin.

### BRIEF DESCRIPTION OF THE DRAWINGS

FIG. 1 is a comparison of BIOD-953, 980 and 981 following accelerated stability conditions of 37° C.+agitation (15 min/day).

FIG. 2 is graph of mean glucagon concentration vs time following intramuscular (IM) injection; it shows baseline subtracted glucagon vs. time in canines. Dose of comparator is 0.5 mg/dog, BIOD-953=1 mg/dog; n=8.

FIG. 3 is a graph of mean glucose concentration vs time following intramuscular (IM) injection; it shows baseline subtracted glucose vs. time in canines. Dose of comparator is 0.5 mg/dog, BIOD-953=1 mg/dog; n=8.

FIG. 4 is a graph of mean glucagon concentration vs. time following IM injection; it shows baseline subtracted glucagon vs. time in canines. Dose of comparator and BIOD-980, 981 is 0.5 mg/dog; n=6.

FIG. 5 is a graph of mean glucose concentration vs time following intramuscular (IM) injection; it shows baseline subtracted glucose vs. time in canines. Dose of comparator and BIOD-980, 981 is 0.5 mg/dog; n=6.

### DETAILED DESCRIPTION OF THE INVENTION

### I. Definitions

As used herein, “glucagon” refers to the full length peptide, glucagon. “GLP-1” refers to glucagon-like peptides (GLP-1, amino acids 7-36 amide and 7-37), and analogs and derivatives thereof, unless otherwise specified.

“Non-aqueous” wherein used herein in connection with a composition refers to compositions containing 5% (w/w) water or less. For example, the composition can contain less than 5%, 4%, 3%, 2.5%, 2%, 1.5%, 0.75%, 0.5%, 0.25%, 0.1, or even 0% water.

“Parenteral administration”, as used herein, means administration by any method other than through the digestive tract or non-invasive topical or regional routes.

“Patient” or “subject” to be treated as used herein refers to either a human or non-human animal.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “stabilize” refers to the reduction or amelioration of the rate, progression, extent and/or duration of degradation, aggregation or denaturation of a protein, or the amelioration of one or more of the effects (preferably, one or more discernible effects) of degradation, aggregation or denaturation of a protein. For example, stabilization may enhance, maintain or prolong the solubility or biological activity of a substance or agent.

“Therapeutically effective” or “effective amount” as used herein means that the amount of the composition used is of sufficient quantity to ameliorate one or more causes or symptoms of a disease or disorder. Such amelioration only requires a reduction or alteration, not necessarily elimination. As used herein, the terms “therapeutically effective amount” “therapeutic amount” and “pharmaceutically effective amount” are synonymous. One of skill in the art can readily determine the proper therapeutic amount.

### II. Compositions

The compositions disclosed include a therapeutically effective amount of glucagon, stabilized as a function of the diluent and antioxidant selected. The diluent and antioxidant are selected to provide a glucagon formulation with increased stability when compared to glucagon without the anti-oxidant and/or the diluent and which shows a comparable onset of action and duration of glucose response.

The disclosed formulation are chemically and physically stable, as measured by their ability to withstand agitation of 15 min per day at 50 revolutions per minute (rpm) using a compact Digital mini rotator (Thermo Scientific) for example, at a temperature ranging from 30° C. to 37° C. for at least 28 days and maintain a glucagon content of >80% of the initial glucagon content, with no gelation. Glucagon content can be determined by reverse phase HPLC (USP method) and gelation can be determined by visual appearance.

The formulations disclosed herein preferably have a glucagon content of >80% at day 7 and at 37° C. For example, the glucagon content can be at 85%, more preferably, at 90%, even more preferably, at 91%, 92%, 93%, 94%, 95%, 96%, 97%, 98% or 99% at day 7 and at 37° C. with agitation. In some preferred embodiments, the glucagon formulations maintain over 80% glucagon content, preferably, over 85%, for example, 86%, 87%, 88%, 89%, 90%, 91%, etc., of its glucagon content at W(week)5. In an even more preferred embodiment, the glucagon formulation maintains over 90% glucagon content at W8 at 30° C. (with agitation), for example, between 90 and 99% of the glucagon content, such as 91%, 92%, 93%, 94%, 95%, 96%, etc., including the intermediate values.

The compositions are pharmacologically equivalent to GLUCAGEN® with respect to AUC (area under the concentration time curve), Cmax, Tmax following intramuscular and/or subcutaneous administration, and provide comparable onset of action and duration of glucose response. A composition is considered to be pharmacologically equivalent to GLUCAGEN® with respect to AUC, Cmax or Tmax, as used herein, when the AUC, Cmax and Tmax of glucagon is in a range of ±15 to 20% of the AUC, Cmax or Tmax obtained with GLUCAGEN® when administered via a similar route to the same subject type. For example, ACU, Cmax or Tmax following subcutaneous administration to a human subject.

A. Glucagon

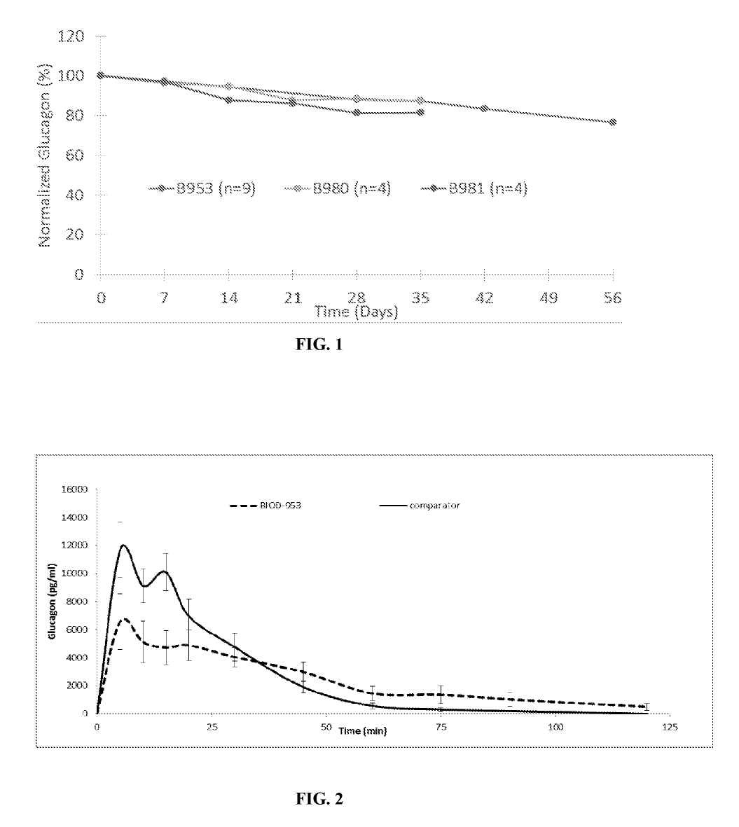

Glucagon is a highly conserved polypeptide consisting of a single chain of 29 amino acids (FIG. 1), with a molecular weight of 3485 Da, synthesized in the pancreas. Recombinant glucagon is expressed in E. coli and purified to at least 95% pure prior to use. Natural and recombinant glucagons are bioequivalent, as demonstrated by Graf, et al., J. Pharm. Sci. 88(10):991-995 (2000). Multiple commercial sources are available. The preferred concentration range for glucagon is 0.5-4 mg/mL, preferably 0.8 to 2.5 mg/mL, most preferably 1-2 mg/mL.

B. Carrier/Solvent

The compositions disclosed herein are non-aqueous. Accordingly, glucagon is mixed with a pharmaceutically acceptable non-aqueous carrier, to provide a composition the water content preferably kept below 5%, preferably below, 3%. A preferred composition has a water content of 2% (i.e., a 2% aqueous composition). Suitable carriers that can be used include, but are not limited to the pharmaceutically acceptable carrier is a non-aqueous carrier including, but are not limited to, lipids, phospholipids, aryl benzonates, alkyl benzonates, triacetin, benzyl benzoate, miglyol. Other suitable non-aqueous solvents include polyethylene glycol (PEG), propylene glycol (PG), polyvinylpyrrolidone (PVP), methoxypropylene glycol (MPEG), glycerol, glycofurol and TC. Additional examples of non-aqueous solvents or vehicles are vegetable oils, such as olive oil and corn oil, gelatin, and injectable organic esters such as ethyl oleate. In a preferred embodiment, the compositions include a mixture of solvents, for example, PG and TC included at a ratio ranging from 1:4 to 4:1, preferably at a ratio of 1:1.

C. Antioxidants

Suitable antioxidants include, but are not limited to, ascorbic acid, cysteine, glutathione, methionine, monothioglycerol, sodium thiosulphate, sulfites, BHT, BHA, ascorbyl palmitate, propyl gallate, butylated hydroxylanisole, butylated hydroxytoluene and Vitamin E. Preferred antioxidants include TPGS (2-20 mg/mL) preferred range 5-10 mg/ml; propyl gallate (C8 and C12 gallate) 0.01-10 mg/ml preferred range 0.5 to 2 mg/ml; butylated hydroxylanisole (0.001-10 mg/ml) preferred range 0.05-0.5 mg/ml, butylated hydroxytoluene (0.001-10 mg/ml) preferred range 0.05-0.5 mg/ml and methionine (0.05-10 mg/mL) preferred range 0.05-4 mg/ml, alone, or in combination.

Kornfelt, et al. (U.S. Pat. No. 5,652,216) discloses acidic pharmaceutical preparations (e.g., pH 2.8) containing glucagon and a pharmaceutically acceptable ampholyte, such as an amino acid (for example, glycine or methionine) or a dipeptide or a mixture thereof. The amount of ampholyte is disclosed between 0.01 to 50 micromoles per mg glucagon in order to stabilize lyophilized glucagon powder, mainly by reducing deamidation and peptide backbone cleavage. The ampholyte molecules alone are not sufficient to suppressed glucagon oxidation in non-aqueous media.

Most preferred formulations are provided in Table 1, all including glucagon at a concentration of 2 mg/ml, and solvent ratio of 1:1.

TABLE 1
Glucagon formulations

| | Anti-oxidant/buffer (NaAC (sodium acetate)) | pH |
|---|---|---|
| BIOD953 | 2% aqueous (methionine, 0.4 mg/ml or 2.68 mM + | 3 |
| | 10 mM NaAC); TPGS, 10 mg/mL | |
| BIOD980 | 2% aqueous (methionine, 0.4 mg/ml or 2.68 mM + | 3 |
| | 10 mM NaAC); Propyl gallate 0.5, mg/mL | |
| BIOD981 | 2% aqueous (methionine, 0.4 mg/ml or 2.68 mM + | 3 |
| | 10 mM NaAC); Butylated hydroxylanisole, 0.1 | |
| | mg/mL | |
| BIOD954.21 | 2% aqueous (methionine, 0.4 mg/ml or 2.68 mM + | 3 |
| | NaAC 10 mM) Butylated Hydroxyltoluene | |
| | 0.1 mg/mL | |

D. Surfactants

In optional embodiments, the compositions include a surfactant. Amphiphilic surfactants (i.e., having at least two positive and two negative charges in different regions of the molecule) such as phospholipids or glycerophospholipids, containing a polar head and two non-polar tails, in combination with sugars are useful in stabilizing the glucagon. These are preferably GRAS (“generally regarded as safe”) phospholipids or endogenous phospholipids. The surfactant may be a sn-glycero-3-phosphate ester of ethanolamine, choline, serine or threonine. Octanoyl, decanoyl, lauroyl, palmitoyl and myristoyl derivatives of lysophosphatidylcholine, lysophosphatidylserine and lysophosphatidylthreonine, are particularly useful.

In a preferred embodiment, the surfactant is LMPC. Surfactant is added in a concentration equivalent to LMPC in a range of 0.1-10 mg/mL, preferably 0.5-5 mg/mL. A preferred concentration is 2 mg surfactant/mL with glucose at 0.25 M.

Surfactant may interact with the glucagon solution to form liposomes. Liposomes (LPs) are spherical vesicles, composed of concentric phospholipid bilayers separated by aqueous compartments. LPs have the characteristics of adhesion to and creating a molecular film on cellular surfaces. Liposomes are lipid vesicles composed of concentric phospholipid bilayers which enclose an aqueous interior (Gregoriadis, et al., Int J Pharm, 300:125-30 (2005); Gregoriadis and Ryman, Biochem J., 124:58P (1971)). The lipid vesicles comprise either one or several aqueous compartments delineated by either one (unilamellar) or several (multilamellar) phospholipid bilayers (Sapra, et al., Curr Drug Deliv., 2:369-81 (2005)). The success of liposomes in the clinic has been attributed to the nontoxic nature of the lipids used in their formulation.

Liposomes have been widely studied as drug carriers for a variety of chemotherapeutic agents (approximately 25,000 scientific articles have been published on the subject). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadl, Curr Med Chem., 12:2193-214 (2005); Tyagi, et al., J Urol, 171, 483-9 (2004)).

The formulation can also be provided as an emulsion, microemulsion (<100 nm) or micelles, formed by addition of water to the surfactant, or surfactant to the water. These embodiments are not preferred for use with a pump or other small orifice means for administration, due to the inherently more viscous nature of liposomes and emulsions. Non-ionic surfactants such as methyl beta cyclodextran or polysorbates (such as TWEEN 20) also may be used to control gelation of the above excipients and/or glucagon.

E. Additional Excipients

The compositions disclosed herein can include additional excipients such as preservatives, osmotic regulators, buffers, and emulsifiers. Exemplary buffers include sodium acetate, citrate and glycine buffers. Suitable preservatives include, but are not limited to, parabens, chlorobutanol, phenol, sorbic acid, and thimerosal. Preservatives such as ethylenediaminetetraacetic acid (“EDTA”), sodium benzoate, metacresol (m-cresol), benzyl alcohol or phenol may also be added to the formulation. Preservatives can be added to a concentration of 0.2 to 10 mg/mL. A preferred concentration of phenol is 2.5-5 mg/ml. A preferred concentration of benzyl alcohol is 5 mg/ml.

Exemplary osmotic regulators are carbohydrate moieties such as monosaccharides or disaccharides. Saccharides can exist in both a straight-chain and cyclic conformation. Simple sugars can stabilize the hydrophilic regions of the polypeptide. Preferred examples include trehalose, lactose, sucrose, maltose or glucose in a concentration range of about 20-100 mg/mL, preferably 0.25 M. In some embodiments glucose is added as an osmotic regulator. Glucose can assist in the elevation of blood sugar on injection. Glucose can be added in a concentration range of 30-60 mg/ml, preferably to a concentration of 45 mg/ml. In other embodiments, glycerin is added as an osmotic regulator. Glycerin can be added in a concentration range of 10-30 mg/ml, preferably to a concentration of 15-22 mg/mL, most preferably 18.9 mg/ml.

### III. Methods of Using

The compositions disclosed herein can be used to treat patients or subjects with very low blood sugar (severe hypoglycemia) that can happen in subjects who have diabetes and use insulin. The compositions are administered parenterally. For example, parenteral administration may include administration to a patient intravenously, intradermally, subcutaneously, intramuscularly by injection, and by infusion.

Pharmacologically, glucagon increases the concentration of glucose in the blood. Six amino acids at the amino-terminus of the glucagon molecule bind specific receptors on liver cells. This leads to an increase in the production of cAMP, which facilitates the catabolism of stored glycogen and increases hepatic gluconeogenesis and ketogenesis. The immediate pharmacologic result is an increase in blood glucose at the expense of stored hepatic glycogen. The onset of action post injection is 5-20 minutes. Glucagon is degraded in the liver, kidney, and tissue receptor sites. Proteolytic removal of the amino-terminal histidine residue leads to loss of the biological activity of glucagon. The half-life of glucagon in plasma is 3 to 6 minutes, similar to that of insulin.

Glucagon can be administered to the subcutaneous tissue as a drug to treat hypoglycemic events. Typically, the dose of glucagon delivered to the subcutaneous tissue will be determined by the needs of the patient. A typical dose of glucagon used to reverse severe hypoglycemic events is 1 mL of a 1 mg/mL solution or equivalent dose at higher concentration.

### Examples

### Example 1. Effect of Varying Ratios of PG:TC and Vitamin E Concentration on the Stability of Glucagon

Formulations in Table 2 were prepared by following procedure: glucagon was dissolved in PG at 4 mg/ml. Then transcutol solution and pre-dissolved Vitamin E (clear solution) were added to the glucagon solution while stirring. Approximately 1 ml of the mixture was added into a 1 ml Uniject. Unijects were then sealed in laminated pouch under nitrogen, and stored in stability chambers.

The formulations made and their appearance is shown in Table 2.

TABLE 2
Initial Stability of glucagon formulations a various
PPG:TC ratios and antioxidant concentrations

| | | Anti- | | |
|---|---|---|---|---|
| | | oxidant | | |
| | Glucagon | Vitamin E | PG:Transcutol | Initial |
| Formulations | (mg/mL) | mg/ml | ratio | Appearance |
| B938.32 | 2 | 1 | 1:1.2 | “clear |
| | | | | looking” |
| B938.33 | 2 | 1 | 1:1 | “clear |
| | | | | looking” |
| B938.34 | 2 | 1 | 1:2 | Haze |
| | | | | suspension |
| B938.36 | 2 | 0.5 | 1:1 | “clear |
| | | | | looking” |
| B938.37 | 2 | 0.5 | 1:1.5 | “translucent |
| | | | | suspension |

“B” as used in Table 2 under “formulations” = BIOD

The formulations in Table 2 were agitated at 50 rpm for 15 min daily at 50 revolutions per minute (rpm) using a Compact Digital Mini Rotator (Thermo Scientific). Samples were stored in 37 C stability chambers and pulled out for 15 min for agitation. Samples were then returned to the chamber for further exposure to 37 C.

Glucagon content was determined by RP-HPLC. The samples were well mixed by vortex before sampling and suspension of solution was dissolved with glucagon diluent. The data is shown in Tables 2 and 3.

The formulations were initially clear looking; however, they changed to suspensions after three days with agitation at 37° C., indicating that particle sizes increased.

TABLE 3
Effect of vitamin E on glucagon content at 37° C.

| | Glucagon | 37° C.-d 7 | |
|---|---|---|---|
| | content | UNIJECT ™ | 37° C.-d 7 |
| | at T₀ | 50 rpm-15 mAgit | UNIJECT ™ |
| Formulations | (mg/ml) | (mg/mL) | 50 rpm-15 mAgit % |
| B938.32 | 1.684 | 1.111 | 65.97 |
| (223-157-B) | | | |
| B938.33 | 1.719 | 1.09 | 63.41 |
| (223-157-B) | | | |
| B938.33 | 1.88 | 1.438 | 76.49 |
| (223-161B) | | | |
| B938.36 | 1.89 | 1.390 | 73.54 |
| (223-161B) | | | |
| B938.37 | 1.83 | 1.481 | 80.93 |
| (223-161B) | | | |

“B” as used in Table 3 under “formulations” = BIOD

The data shows significant loss (>20%) of glucagon at day 7, at 37° C. due to oxidation [based on the degradation products identified by HPLC chromatograms]. Glucagon loss was observed regardless of whether 0.5 mg/Ml or 1 mg/ml vitamin E was used.

### Example 2. Effect of Various Antioxidant Agents on the Stability of Glucagon at a PG:TC of 1

Various glugacon formulations were prepared using different antioxidants alone or in combination as shown in Table 4.

TABLE 4
Glucagon compositions with various antioxidants and surfactants

| | | Surfactant, | |
|---|---|---|---|
| | Antioxidant | mg/ml | pH |
| B938.39 | N/A | N/A | N/A (fully non- |
| | | | aqueous) |
| B938.43 | N/A | A3, 2 | N/A (fully non- |
| | | | aqueous) |
| B938.44 | 2% water | A3, 2 | 5.7 |
| B938.45 | 2% methionine solution | A3, 2 | 5.8 |
| B938.46 | 2% methionine solution + | A3, 2 | 5.8 |
| | EDTA (19.6 μM) | | |
| B938.47 | 2% methionine solution | A3, 2 | 5.8 |
| | Vitamin E (0.5 mg/ml) | | |
| B938.48 | 2% methionine solution + | TPGS, 10 | 5.8 |
| | EDTA (19.6 μM) + TPGS | | |
| B938.49 | 2% methionine solution | A3, 10 | 5.8 |
| B938.50 | % methionine solution | A3, 2 | 3.8 |
| | 5.6 mg/mL citric acid | | |
| B938.51 | 2% methionine buffer | A3, 2 | 6.8 |
| | solution | | |

A3 (n-Dodecyl β-D-maltoside)
“B” as used in Table 4 under “formulations” = BIOD

The formulations in Table 4 were stored at 37° C. and agitated at 50 rpm for 15 min daily at and the effect on glucagon content measured. The data is shown in Table 5.

TABLE 5
Effect of antioxidants on glucagon stability as a function of time

| | | T0 | D 3 | D 7 | D 14 | D 28 | |
|---|---|---|---|---|---|---|---|
| | | mg/ | mg/ | mg/ | mg/ | mg/ | D 28 |
| Lot# | Code | mL | mL | mL | mL | mL | % |
| 266-36- | B938.39 | 1.913 | 1.492 | 0.890 | 0.298 | N/A* | N/A* |
| 092614 | (control) | | | | | | |
| (B) | B938.43 | 1.965 | 1.683 | 1.536 | 1.150 | N/A* | N/A* |
| | B938.44 | 1.962 | 1.757 | 1.669 | 1.508 | N/A* | N/A* |
| | B938.45 | 1.990 | 1.816 | 1.726 | 1.595 | N/A* | N/A* |
| 266-39- | B938.45 | 2.056 | 1.86 | 1.776 | 1.627 | 1.284 | 62.45 |
| 10314 | (control) | | | | | | |
| (B) | B938.46 | 1.957 | 1.44 | 0.718 | 0.255 | N/A** | N/A** |
| | B938.47 | 2.024 | 1.80 | 1.764 | 1.654 | 1.613 | 79.69 |
| | B938.48 | 2.044 | 1.87 | 1.795 | 1.794 | 1.576 | 77.10 |
| | B938.49 | 1.895 | 1.72 | 1.488 | 1.347 | 1.106 | 58.36 |
| | B938.50 | 1.71 | 1.375 | 1.191 | 0.184 | N/A** | N/A** |
| | B938.51 | 1.865 | 1.71 | 1.661 | 1.440 | 1.184 | 63.55 |

*unavailable due to data design
**unavailable due to poor performance of previous time point
“B” as used in Table 5 under “formulations” = BIOD

At day 28, the glucagon content for B938.45 (control) was only at 62.45% of the content at the beginning of the experiment (T₀). Increasing the content of A3 to 10 mg/ml did not improve this loss in glucagon content (B938.49), neither did raising the pH from 5.8 to 6.8 (B938.51). A combination of anti-oxidant agents reduced oxidation, especially B938.47 (methionine and vitamin E) & B938.48 (methionine+EDTA+TPGS) when compared to the control formulation (B938.45).

All the formulations except BIOD-938.50 in Table 5 became a suspension by the end of week 2 at 37° C. Low pH seems to be able to control the particle size of glucagon (dynamic light scattering, (Malvern)). BIOD-938.50 (2% methionine solution, A3 (2 mg/ml) at a pH of 3.8), remained a clear solution throughout the test although the remaining glucagon content was low at 37° C. Therefore, a better anti-oxidant is needed to restore the glucagon content.

### Example 3

Formulations were prepared as shown in Table 6 to test the effect of water concentration on glucagon stability.

TABLE 6
Glucagon formulations with varying water concentrations

| | | | | | | | | Normalized |
|---|---|---|---|---|---|---|---|---|
| | | | Glucagon content (mg/ml) | | | | | Glucagon |
| pH | lot 266-42- | % | | ³⁷° C.- | 37° C.- | 37° C.- | 37° C.- | content % |
| (surfactant) | 100814 | water | T₀ | d 3 | d 7 | D 14 | D 28 | D 28% |
| 3 | B938.53 | 2 | 2.051 | 2.011 | 1.885 | 1.791 | 1.628 | 79.381 |
| 3 | B938.59 | 5 | 2.095 | 2.124 | *2.066 | *1.922 | *1.685, | 73.27 |
| | | | | | | | 1.380 | |
| 3 | B938.62- | 10 | 2.055 | *2.118 | *2.072 | *1.794 | *1.788, | 93.67 |
| | | | | | | | 2.065 | |
| 3 | B938.58- | 5 | 2.000 | 1.998 | 1.948 | *1.905 | *1.745 | 87.64 |
| 4 | B938.54 | 2 | 2.052 | 2.084 | 2.013 | 1.888 | 1.849 | 90.11 |
| 5.8 | B938.48 | 2 | 2.038 | 1.904 | 1.600 | 1.735 | 1.624 | 79.69 |
| 8(TPGS) | B938.52 | 2 | 2.055 | 2.019 | 1.923 | 1.871 | 1.755 | 85.40 |
| 3 (A3) | B938.63- | 2 | 2.005 | 1.910 | 1.565 | 1.236 | 0.945 | 47.13 |
| 8(A3) | B938.64 | 2 | 2.076 | 2.053 | 1.957 | 1.907 | 1.784 | 85.93 |
| | B938.54 ) | 2 | 1.948 | 1.943 | 1.96 | 1.864 | N/A | N/A |
| | B938.65 | 5 | 1.984 | *1.862 | *0.62 | *0.968 | N/A | N/A |
| | B938.66-( ) | 5 | 4.436 | *3.707 | *1.87 | *2.363 | N/A | N/A |
| | B938.56 | 5 | 3.992 | *2.413, | *1.61, | 2.710 | 2.996 | 75.05 |
| | | | | 3.343 | 0.4 | | | |
| | B938.57 | 5 | 4.005 | *2.962 | *0.996 | *1.722 | N/A | N/A |

*gelled samples, which can have high variable data.
two data point indicate values obtained for two samples, but data not close to each other unlike other points, which showed the average of two samples
N/A indicates data could not be generated: terminated the test due poor data of previous time point
Therefore, water content ≧5% created gelling with either 2 or 4 mg/ml glucagon. Preferable to keep water 2% or less.
“B” as used in Table 6 under “formulations” = BIOD

### Example 4. The Effect of Varying Concentrations of TPGS, Percentage Water, pH and/or Different Buffers on Glucagon Stability

Glucagon formulations were prepared containing glucagon at 2 mg/ml, and various water, pH and TPGS concentrations as shown in Table 7. The formulations were prepared by following procedure: Glucagon was dissolved in acidified PG or alkaline PG at 4 mg/mL depending the final pH of formulations, then added 20 mg/mL methionine stock solution in a buffer, stirring to mix; TPGS stock solution was added in Transcutol, stirring to mix, adjusted pH to target, continued stirring for 10 min or till well mixed, filtered via 0.2 membrane under vacuum, filled into Unijects as described earlier. The buffers used in the formulations tested included NaAC, glycine, tris and citric acid.

TABLE 7
Glucagon formulations with varying concentrations
of TPGS, % water, pH and buffers

| | | Surfactant | | |
|---|---|---|---|---|
| Lot# 266-86- | | (TPGS) | | Glucagon |
| 120914 | Anti-oxidants/buffer | mg/ml | pH | mg/ml |
| BIOD-938.54 | 2% aqueous (methionine, | 10 | 4 | 2 |
| (Control) | 0.4 mg/ml final) + NaAC | | | |
| | (10 mM) | | | |
| BIOD-953 | 2% aqueous (methionine, | 10 | 3 | 2 |
| (control) | 0.4 mg/ml final) + NaAC | | | |
| | (10 mM) | | | |
| BIOD-938.79 | 2% aqueous (methionine, | 5 | 3 | 2 |
| | 0.4 mg/ml final) + NaAC | | | |
| | (10 mM) | | | |
| BIOD-938.80 | 2% aqueous (methionine, | 2 | 3 | 2 |
| | 0.4 mg/ml final) + NaAC | | | |
| | (10 mM) | | | |
| BIOD-938.81 | 2% aqueous (methionine, | 1 | 3 | 2 |
| | 0.4 mg/ml final) + NaAC | | | |
| | (10 mm) | | | |
| BIOD-938.82 | 2% aqueous (methionine, | 20 | 3 | 2 |
| | 0.4 mg/ml final) + NaAC | | | |
| | (10 mM) | | | |
| BIOD-938.83 | 1% aqueous (methionine, | 2 | 3 | 2 |
| | 0.4 mg/ml final) + NaAC | | | |
| | (10 mM) | | | |
| BIOD-938.87 | 3% aqueous (methionine, | 2 | 3 | 2 |
| | 0.4 mg/ml final) + NaAC | | | |
| | (10 mM) | | | |
| BIOD-938.88 | 2% aqueous (methionine, | 2 | 2 | 2 |
| | 0.4 mg/ml final) + NaAC | | | |
| | (10 mM) | | | |
| BIOD-938.84 | 2% aqueous (methionine, | 2 | 3 | 2 |
| | 0.8 mg/ml final) + NaAC | | | |
| | (10 mM) | | | |
| BIOD-938.85 | 2% aqueous (methionine, | 2 | 3 | 2 |
| | 0.4 mg/ml final) + | | | |
| | citric acid 10 mM) | | | |
| BIOD-938.86 | 2% aqueous (methionine, | 2 | 3 | 2 |
| | 0.4 mg/ml final) + glycine | | | |
| | 10 mM) | | | |
| BIOD-938.89 | 2% aqueous (methionine, | 2 | 9 | 2 |
| | 0.4 mg/ml final) + | | | |
| | tris 10 mM | | | |

The formulations in Table 7 were clear throughout the test. The stability data for formulations shown in Table 7 is provided below in Table 8.

TABLE 8
Glucagon content in formulations containing varying concentrations of TPGS

| lot | | | | | | (%)* | | | (%) |
|---|---|---|---|---|---|---|---|---|---|
| 266-86-120914 | | 37° C.- | 37° C.- | 37° C.- | 37° C.- | 37° C.- | 30 C.- | 30° C.- | 30° C.- |
| (A) | T0 | W 1 | W 2 | W 4 | 5 W | W 5 | W 4 | W 8 | W 8 |
| BIOD-953 (control, | 1.937 | 1.894 | 1.826 | 1.775 | 1.761 | 90.91 | 1.864 | 1.786 | 92.20 |
| 10 mg/mL TPGS) | | | | | | | | | |
| BIOD-938.54 | 1.906 | 1.870 | 1.832 | 1.760 | 1.723 | 90.40 | 1.845 | 1.754 | 92.03 |
| (control) | | | | | | | | | |
| BIOD-938.79 | 1.922 | 1.853 | 1.830 | 1.747 | 1.732 | 90.11 | 1.849 | 1.757 | 91.42 |
| (5 mg/mL TPGS) | | | | | | | | | |
| BIOD-938.80 | 1.869 | 1.822 | 1.786 | 1.719 | 1.678 | 89.78 | 1.790 | 1.710 | 91.49 |
| (2 m/mL | | | | | | | | | |
| TPGS) = B955 | | | | | | | | | |
| BIOD-938.81 | 1.942 | 1.894 | 1.855 | 1.788 | 1.734 | 89.29 | 1.874 | 1.780 | 91.66 |
| (1 mg/mL TPGS) | | | | | | | | | |
| BIOD-938.82 | 1.967 | 1.914 | 1.887 | 1.809 | 1.771 | 90.04 | 1.906 | 1.809 | 91.97 |
| (20 mg/ml TPGS) | | | | | | | | | |
| BIOD-938.83 | 1.929 | 1.861 | 1.829 | 1.747 | 1.705 | 88.39 | 1.864 | 1.750 | 90.72 |
| (1% Aqueous) | | | | | | | | | |
| BIOD-938.84 | 1.917 | 1.882 | 1.832 | 1.764 | 1.724 | 89.93 | 1.855 | 1.768 | 92.23 |
| (0.8 mg/mL | | | | | | | | | |
| Methionine) | | | | | | | | | |
| BIOD-938.87 | 1.952 | 1.924 | 1.879 | 1.786 | 1.754 | 89.86 | 1.886 | 1.810 | 92.73 |
| (3% Aqueous) | | | | | | | | | |
| BIOD-938.88 | 1.959 | 1.801 | 1.691 | 1.488 | 1.359 | 69.37 | 1.702 | 1.498 | 76.47 |
| (pH 2) | | | | | | | | | |
| BIOD-938.85 | 1.964 | 1.910 | 1.843 | 1.740 | 1.678 | 85.44 | 1.847 | 1.749 | 89.05 |
| (citric acid) | | | | | | | | | |
| BIOD-938.86 | 1.973 | 1.929 | 1.859 | 1.752 | 1.692 | 85.76 | 1.860 | 1.763 | 89.36 |
| (Glycine) | | | | | | | | | |
| BIOD-938.89 | 1.823 | 1.747 | 1.700 | 1.584 | 1.534 | 84.15 | 1.696 | 1.581 | 86.73 |
| (pH 9) | | | | | | | | | |

Conclusions:
1. Order of pH effect on stability pH 3-4 < pH 9 < pH 2
2. Buffers slightly impact on stability citric > glycine > acetate;
3. Water concentration at 1-3% was same.
4. No difference in effect was observed at the TPGS concentrations used

### Example 5. The Effect of Substituting TPGS with Other Antioxidants on Glucagon Stability

Hydrophobic anti-oxidants such propyl gallate (PG,BIOD-980), BHA(BIOD-981), BHT (BIOD-0954.21) were tested to determine whether they can stabilize glucagon and improve the pK profiles. Stability result indicated they are as effective as TPGS in combination with methionine to suppress glucagon oxidation. The new antioxidants also showed better absorption, displaying a greater Cmax and AUC (area under the concentration time curve) than that seen with TPGS.

Various glucagon formulations were prepared as shown in Table 1, in which propyl gallate and butylated hydroxylanisole were used to provide antioxidant properties. The stability data for these formulations is shown in

TABLE 9
Glucagon content in formulations containing varying antioxidants

| | Normalized Glucagon (%) | | | | | | | |
|---|---|---|---|---|---|---|---|---|
| Formulations | Time 0 | Day 7 | Day 14 | Day 21 | Day 28 | Day 35 | Day 42 | Day 56 |
| Lilly glucagon | 100 | 74.35 ± 2.53** | | | | | | |
| (n = 2) | | (day 6) | | | | | | |
| (reconstituted) | | | | | | | | |
| BIOD-954 | 100 | 76.41 ± 16.4 | 53.50 ± 32.66 | N/A* | 34.03 ± 30.84 | N/A | | |
| (n = 9) | | | | | | | | |
| BIOD-953 | 100 | 97.30 ± 1.08 | 94.61 ± 0.99 | N/A | 87.97 ± 6.11 | 87.30 ± 2.86 | 83.48 ± 1.43 | 76.55 ± 5.71 |
| (n = 9) | | | | | | | | (n = 2) |
| BIOD-980 | 100 | 96.46 ± 1.10 | 94.81 ± 1.40 | 87.80 ± 3.94 | 88.50 ± 2.41 | 86.99 ± 0.54 | | |
| (n = 4) | | | | | | | | |
| BIOD-981 | 100 | 96.86 ± 1.58 | 87.57 ± 9.18 | 86.12 ± 4.67 | 81.32 ± 5.62 | 81.61 ± 5.10 | | |
| (n = 4) | | | | | | | | |
| BIOD954.21 | 100 | 97.2 | 93.5 | 89.4 | 83.9 | N/A* | | |

Conclusion: There is improved stability with PG, BHT and BHA

### Example 6. Example of Improvement in Pharmacokinetics (PK) Absorption by Substitution of Anti-Oxidant

Basic formulation: PG/transcutol 1:1; Composition of BIOD-953: 2% water, methionine, 0.4 mg/ml or 2.68 mM+NaAC 10 mM+Tocopheryl polyethylene glycol succinate (TPGS) 10 mg/mL.

The stability of this formulation is acceptable, achieving greater than 80% glucagon content in excess of 28 days at 37 C. However, twice the amount of glucagon must be given to achieve equivalent dose of comparator (commercially available glucagon kit).

Exchanging the TPGS for 0.5 mg/ml Propyl gallate (BIOD-980) or 0.1 mg/ml butylated hydroxylanisole (BIOD-981), more efficient absorption may be achieved. Chemical stability of all formulations remains comparable across these formulations (FIG. 1).

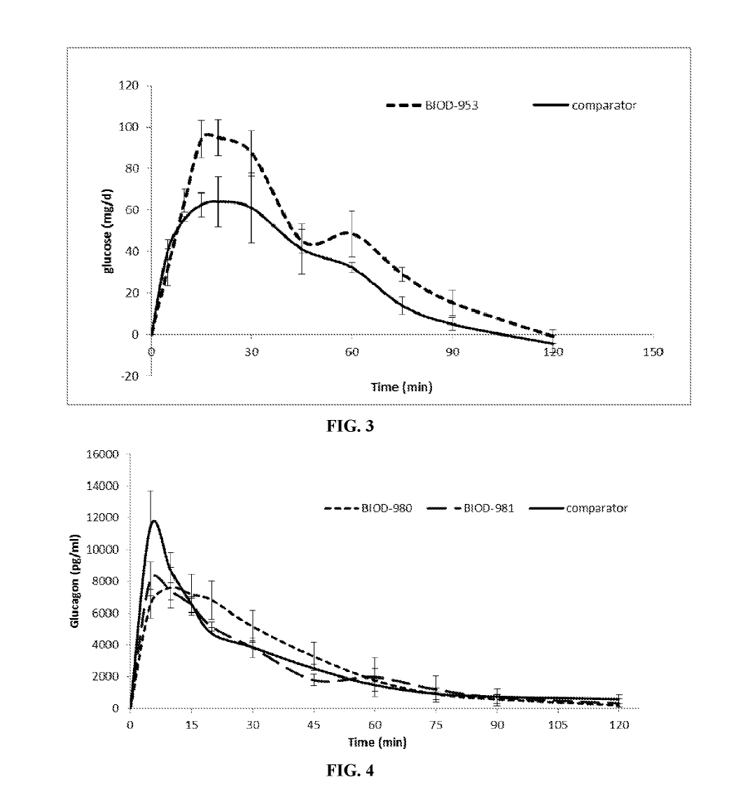

A PK/PD study was done in canine, using 8 animals and dosing in a cross over design. Dogs were fed twice their normal canine diet the day prior to dosing to assure sufficient glycogen stores. On the morning of the study, the animals were fasted and given either an IM dose of BIOD-953 (0.5 mg/dog) or a 0.5 mg dose of freshly reconstituted glucagon from a commercially supplied glucagon kit. The animals were studied over two hours and plasma samples were taken at −10, 0, 5, 10, 15, 20, 30, 45, 60, 75, 90 and 120 min. post dose. Plasma was analyzed for glucagon content (ELISA) and glucose concentration (YSI glucose analysis). Comparable areas under the curve were obtained and timing of both formulations was similar following intramuscular (IM) injection (FIGS. 2 and 3).

In another study, 6 canines were dosed with either BIOD-980, 981 or comparator; dose 0.5 mg/dog. The study was otherwise conducted as described above.

PK parameters from this study are shown below in Table 10.

TABLE 10
Pharmacokinetic parameters for formulations 953, 980 and 981

| | BIOD-953 | BIOD-980 | BIOD-981 |
|---|---|---|---|
| | Vs. | Vs. | Vs. |
| Variable | Comparator | Comparator | Comparator |
| Cₘₐₓ | 7386 ± 1769 Vs. | 8518 ± 1202 Vs. | 8927 ± 782 Vs. |
| (pg/mL) | 12741 ± 1668 | 11831 ± 1468 | 11831 ± 1468 |
| | (42% ↓) | (28% ↓) | |
| Tₘₐₓ | 13 ± 2 Vs. | 16 ± 4 Vs. | 8 ± 2 Vs. |
| (minutes) | 11 ± 1 | 8 ± 2 | 8 ± 2 |
| | (18% ↓) | | |
| AUC_{0-120 min} | 287552 ± 59805 | 328036 ± 64748 | 276870 ± 47136 |
| (pg/mL*min) | Vs. | Vs. | Vs. |
| | 312042 ± 46056 | 299986 ± 19333 | 299986 ± 19333 |

*= value has been divided by 2 (dose 1 mg/dog) so it may be compared to other formulations at 0.5 mg/dog.

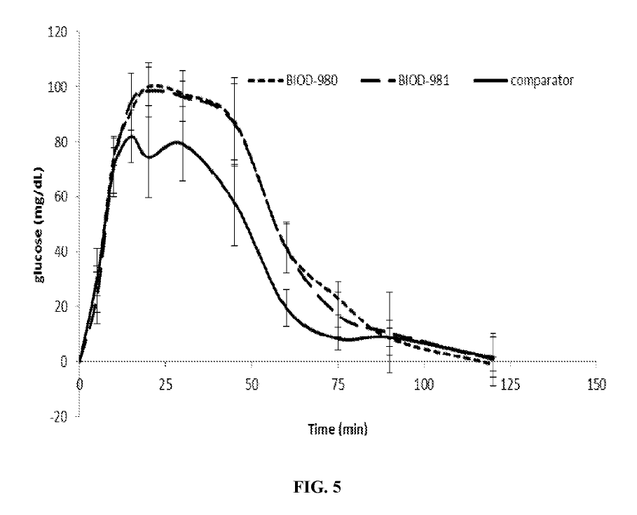

The data is depicted graphically for BIOD-980 and BIOD-981 in FIGS. 4 and 5 for glucagon and glucose levels, respectively. BIOD-953 is in FIGS. 2 and 3.

Conclusions: By exchanging the anti-oxidant, there is an improvement in the amount of glucagon absorbed, AUC (0-120 min), thereby allowing equivalent doses to be given to achieve similar PK profiles.

I claim:

1. A stabilized glucagon formulation comprising a pharmaceutically acceptable antioxidant in a non-aqueous diluent effective to stabilize the glucagon.

2. The formulation of claim 1, wherein the antioxidant is selected from the group consisting of propyl gallate, butylated hydroxylanisole, butylated hydroxytoluene, d-α-tocopheryl polyethylene glycol 1000 succinate (TPGS), methionine and combinations thereof.

3. The formulation of claim 1, comprising TPGS in a concentration ranging from 2-20 mg/ml.

4. The pharmaceutical preparation according to claim 1, further comprising a pharmaceutical acceptable excipient.

5. The formulation of claim 1, comprising water in an amount ranging from 0 to 5%.

6. The formulation of claim 5, comprising water in an amount ranging from 1-3%.

7. The formulation of claim 1, wherein the non aqueous diluent is selected from the group consisting of propylene glycol (“PG”) and diethylene glycol monoethyl ether (TRANSCUTOL® (“TC”)).

8. The formulation of claim 7, wherein the ratio of PG:TC in the formulation is from 1:4 to 4:1.

9. The formulation of claim 8 wherein the ratio of PG:TC in the formulation is 1:1.

10. The formulation of claim 2, comprising propyl gallate in a concentration ranging from 0.01-10 mg/ml.

11. The formulation of claim 2, comprising methionine in a concentration range between 0.05-10 mg/ml.

12. The formulation of claim 1 comprising glucagon in a concentration ranging from 0.4-4 mg/ml.

13. The formulation of claim 12, wherein the pH of the formulation is in a range from 2-4 or 8-9.

14. The formulations of claim 1, wherein the formulation maintain at least 80% glucagon content as measured by reverse phase high performance liquid chromatography following agitation of 15 min per day at 50 revolutions per minute (rpm) at least 7 days.

15. The formulation of claim 14 wherein the formulation maintains at least 80% glucagon content following agitation of 15 min per day at 50 revolutions per minute (rpm) at least 28 days.
